(19) United States
(12) Patent Application Publication
(10) Pub. No.: US 2013/0172585 A1
(43) Pub. Date: Jul. 4, 2013

(54) PROCESS FOR THE PRODUCTION OF FURFURAL

(71) Applicant: E I DU PONT DE NEMOURS AND COMPANY, Wilimington, DE (US)

(72) Inventors: Stuart B. Fergusson, Kingston (CA); Michael Stephen Mckinnon, Kingston (CA); Ronnie Ozer, Arden (DE)

(73) Assignee: E I DU PONT DE NEMOURS AND COMPANY, Wilmington, DE (US)

(21) Appl. No.: 13/729,559

(22) Filed: Dec. 28, 2012

Related U.S. Application Data
(60) Provisional application No. 61/580,739, filed on Dec. 28, 2011.

(51) Int. Cl. C07D 307/50

(52) U.S. Cl. CPC C07D 307/50
USPC 549/489

(57) ABSTRACT

Furfural is produced by contacting an aqueous feedstock solution containing C₅ sugar and/or C₆ sugar using a soluble acid catalyst with reactive distillation. Both high yield and high conversion are obtained in the reaction vessel. Degradation of furfural is minimized by its low residence time in contact with the acid catalyst. The use of staged distillation improves the furfural yield.

### CROSS-REFERENCE TO RELATED APPLICATIONS

This application claims the benefit under 35 U.S.C. §119(e) of U.S. Provisional Application No. 61/508,739, filed Dec. 28, 2011 which is herein incorporated by reference.

### FIELD OF THE INVENTION

A method for the production of furfural and related compounds from sugar streams is provided.

### BACKGROUND OF THE INVENTION

Furfural and related compounds, such as hydroxymethylfurfural (HMF), are useful precursors and starting materials for industrial chemicals for use as pharmaceuticals, herbicides, stabilizers, and polymers. The current furfural manufacturing process utilizes biomass such as corn cob, switchgrass or wood waste as a raw material feed stock for obtaining xylose or hemicellulose.

The hemicellulose is hydrolyzed under acidic conditions to its monomer sugars, such as glucose, fructose, xylose, mannose, galactose, rhamnose, and arabinose. Xylose, which is a pentose (i.e., a “C₅ sugar”) is the sugar present in the largest amount. In a similar aqueous acidic environment, the C₅ sugars are subsequently dehydrated and cyclized to furfural. The rate of dehydration is an order of magnitude slower than hydrolysis.

A major difficulty with known methods for dehydration of sugars is the formation of undesirable resinous material that not only leads to yield loss but also leads to fouling of exposed reactor surface and negatively impacts heat transfer characteristics.

There remains a need for a process to produce furfural and related compounds from sugars at both high yield and high conversion.

### SUMMARY OF THE INVENTION

In an aspect of the invention, there is a process comprising:
(a) providing a reactor comprising a reactive distillation column disposed on top of a reboiler and an aqueous acid solution disposed in a reaction zone of the reboiler;
(b) bringing an aqueous feedstock solution into contact with the aqueous acid solution in the reaction zone for a residence time sufficient to produce a mixture of water and furfural, wherein the aqueous feedstock solution comprises C₅ sugar, C₆ sugar or a mixture thereof, and the reaction zone is at a temperature in the range of 90-250° C. and a pressure in the range of 0.1-3.87 MPa;
(c) removing the mixture of water and furfural from the top of the reactive distillation column; and
(d) collecting water, unreacted sugars and nonvolatile byproducts from the reboiler.

In another aspect, there is a process comprising the steps of:
(a) providing a reactor comprising a multistage reactive distillation column comprising an upper, rectifying section; a lower, stripping section; and a reboiler, wherein the reboiler is a reaction zone containing an aqueous acid solution;
(b) continuously feeding an aqueous feedstock sugar solution comprising C₅ sugar, C₆ sugar or a mixture thereof to the reboiler, or to the column at any point, into contact with the aqueous acid solution, thereby forming a reaction mixture, wherein
   (i) the temperature of the reaction mixture is between about 90° C. and about 250° C.,
   (ii) the reaction mixture is held at a pressure between about atmospheric pressure and about 400 psig, and
   (iii) the feedstock sugar solution and aqueous acid catalyst solution are in contact for a time sufficient to produce water and furfural;
(c) drawing off a mixture of furfural and water at the top of the column;
(d) collecting water, unreacted sugars, and nonvolatile byproducts in the reboiler;
(e) removing nonvolatile byproducts from the reboiler; and
(f) removing the water and unreacted sugars from the reboiler for further use or disposal.

### BRIEF DESCRIPTION OF THE DRAWINGS

Various features and/or embodiments of this invention are illustrated in drawings as described below. These features and/or embodiments are representative only, and the selection of these features and/or embodiments for inclusion in the drawings should not be interpreted as an indication that subject matter not included in the drawings is not suitable for practicing the invention, or that subject matter not included in the drawings is excluded from the scope of the appended claims and equivalents thereof.

FIG. 1 is a schematic illustration of an exemplary reactor configuration used in the production of furfural in accordance with various embodiments of the present invention.

FIG. 2 is a schematic illustration of another exemplary reactor configuration used in the production of furfural in accordance with various embodiments of the present invention.

### DETAILED DESCRIPTION

### Definitions

The methods described herein are described with reference to the following terms.

As used herein, the term “sugar” includes monosaccharides, disaccharides, and oligosaccharides. Monosaccharides, or “simple sugars,” are aldehyde or ketone derivatives of straight-chain polyhydroxy alcohols containing at least three carbon atoms. A pentose is a monosaccharide having five carbon atoms; some examples are xylose, arabinose, lyxose and ribose. A hexose is a monosaccharide having six carbon atoms; some examples are glucose and fructose. Disaccharide molecules (e.g., sucrose, lactose, fructose, and maltose) consist of two covalently linked monosaccharide units. As used herein, “oligosaccharide” molecules consist of about 3 to about 20 covalently linked monosaccharide units.

As used herein, the term “Cₙ, sugar” includes monosaccharides having n carbon atoms; disaccharides comprising monosaccharide units having n carbon atoms, and oligosaccharides comprising monosaccharide units having n carbon atoms. Thus, “C₅ sugar” includes pentoses, disaccharides comprising pentose units, and oligosaccharides comprising pentose units.

As used herein, the term “hemicellulose” refers to a polymer comprising C₅ and C₆ monosaccharide units. Hemicellulose consists of short, highly branched chains of sugars. In contrast to cellulose, which is a polymer of only glucose, a hemicellulose is a polymer of five different sugars. It contains five-carbon sugars (usually D-xylose and L-arabinose) and six-carbon sugars (D-galactose, D-glucose, and D-mannose, fructose). Hemicellulose can also contain uronic acid, sugars in which the terminal carbon's hydroxyl group has been oxidized to a carboxylic acid, such as, D-glucuronic acid, 4-O-methyl-D-glucuronic acid, and D-galacturonic acid. The sugars are partially acetylated. Typically the acetyl content is 2 to 3% by weight of the total weight of the hemicellulose. Xylose is typically the sugar monomer present in hemicellulose in the largest amount.

As used herein, the term “organic acid” means an organic compound having acidic properties; some examples are acetic acid, formic acid, and methane sulfonic acid.

As used herein, the term “mineral acid” means an inorganic acid, as distinguished from organic acid. Some examples are sulfuric acid, nitric acid, phosphoric acid, and hydrochloric acid.

As used herein, the term “heteropolyacid” denotes an oxygen-containing acid with P, As, Si, or B as a central atom which is connected via oxygen bridges to W, Mo or V. Some examples are phosphotungstic acid, molybdophosphoric acid.

As used herein, the term “nonvolatile byproduct” denotes a reaction byproduct that either has a boiling point at one atmosphere greater than the boiling point of the distilled product(s), or is a nonvolatile solid.

As used herein the term “humin(s)” refers to dark, amorphous byproduct(s) resulting from acid induced sugar and furfural degradation.

As used herein, the term “selectivity” refers to the moles of furfural produced, divided by the moles of xylose transformed to products over a particular time period.

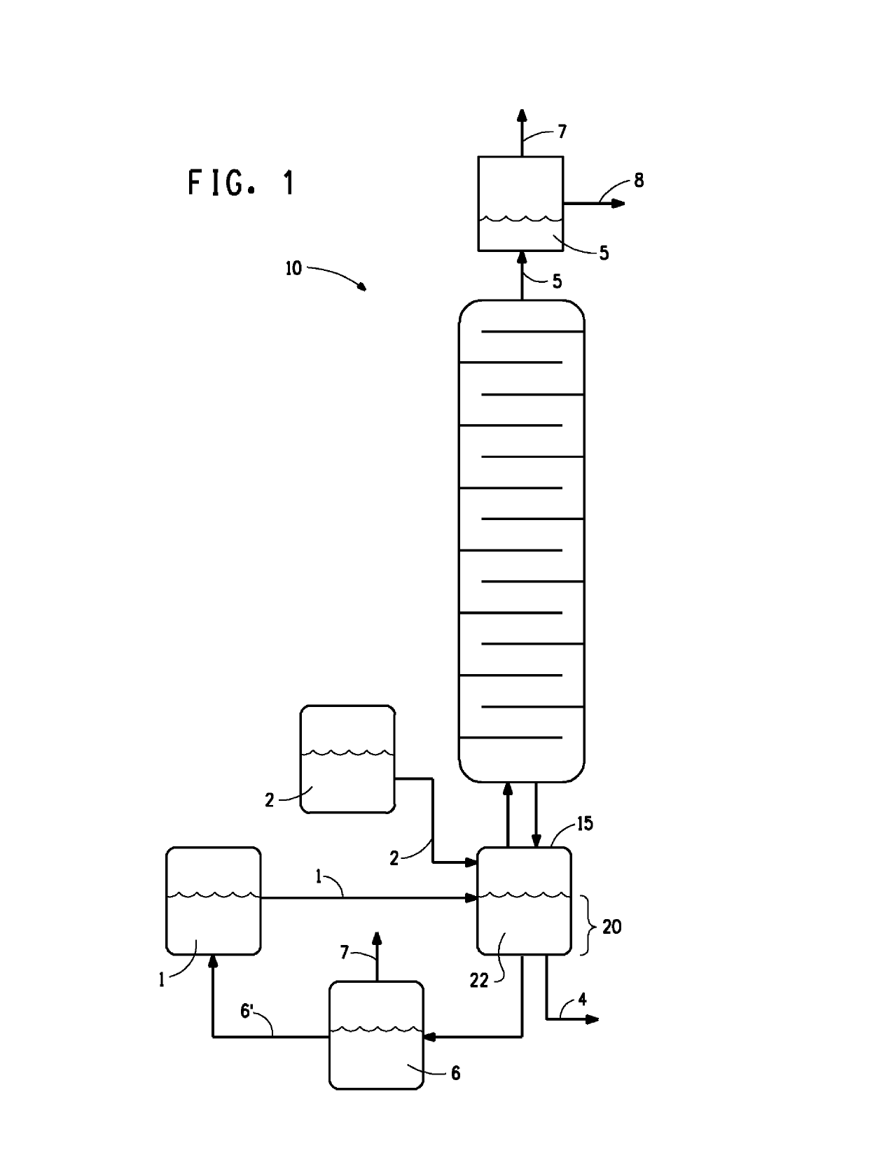

In an embodiment, there is a process for the production of furfural comprising providing a reactor comprising a reactive distillation column disposed on top of a reboiler and an aqueous acid solution disposed in a reaction zone of the reboiler. FIG. 1 shows a schematic illustration of an exemplary reactor configuration comprising a reactive distillation column 10 disposed on top of a reboiler 15 and an aqueous acid solution 2 disposed in a reaction zone 20 of the reboiler 15. In one configuration, the reboiler is a continuous stirred-tank reactor (CSTR).

The aqueous acid solution comprises a mineral acid, a heteropolyacid, an organic acid, or a combination thereof. In one embodiment, the acid catalyst is a mineral acid comprising sulfuric acid, phosphoric acid, hydrochloric acid, or a combination of these. In another embodiment, the acid catalyst is a heteropolyacid comprising phosphotungstic acid, molybdophosphoric acid, or a combination of these.

In other embodiment, the acid catalyst is an organic acid comprising oxalic acid, formic acid, acetic acid, an alkyl sulfonic acid, an aryl sulfonic acid, a halogenated acetic acid, a halogenated alkylsulfonic acid, a halogenated aryl sulfonic acid, or a combination of these. An example of a suitable alkyl sulfonic acid is methane sulfonic acid. An example of a suitable aryl sulfonic acid is toluenesulfonic acid. An example of a suitable halogenated acetic acid is trichloroacetic acid. An example of a suitable halogenated alkylsulfonic acid is 1,1,2,2-tetrafluoroethanesulfonic acid. An example of a suitable halogenated aryl sulfonic acid is fluorobenzenesulfonic acid.

The acid is present in the aqueous acid solution in the range of 0.01-20 or 0.01-5 or 0.1-1.5 weight % based on the total weight of the acid solution (water and acid). In some embodiments, the acid is present in the solvent at a weight percentage between and optionally including any two of the following values: 0.01, 0.05, 0.10, 0.15, 0.20, 0.50, 1.0, 1.5, 2.0, 3.0, 4.0, 5.0, 6.0, 7.0, 8.0, 9.0, 10, 12, 14, 16, 18 and 20 weight percent. The optimal amount of acid catalyst will be affected by the specific reaction conditions and is readily determined by one of skill in the art.

The process for the production of furfural also comprises, as shown in the FIG. 1, bringing an aqueous feedstock solution 1 into contact with the aqueous acid solution 2 to form a reaction mixture 22 in the reaction zone 20 for a residence time sufficient to produce a mixture 5 of water 7 and furfural 8. In an embodiment, the aqueous feedstock solution comprises C₅ sugar, C₆ sugar or a mixture thereof. In another embodiment, the reaction zone 20 is at a temperature in the range of 90-250° C. and a pressure in the range of 0.1-3.87 MPa.

The aqueous feedstock solution comprises at least one C₅ sugar, at least one C₆ sugar, or a mixture of at least one C₅ sugar, and at least one C₆ sugar. Examples of suitable C₅ sugars (pentoses) include without limitation xylose, arabinose, lyxose and ribose. Examples of suitable C₆ sugars (hexoses) include without limitation glucose, fructose, mannose, and galactose.

In one embodiment, the aqueous feedstock solution comprises xylose. In other embodiment, the aqueous feedstock solution comprises glucose. In another embodiment, the aqueous feedstock solution comprises xylose and glucose

The total amount of sugar (C₅ sugar, C₆ sugar, or a mixture thereof) present in the aqueous feedstock solution is in the range of at 0.1-99 weight % or 1-50 weight % or 5-35 weight % or 5-10 weight % based on the total weight of the aqueous feedstock solution. In some embodiments, the C₅ sugar is present in the feedstock solution at a weight percentage between and optionally including any two of the following values: 0.1, 0.5, 1, 2, 3, 4, 5, 6, 7, 8, 9, 10, 15, 17, 19, 21, 23, 25, 30, 35, 40, 45, 50, 55, 60, 65, 70, 75, 80, 85, 90, 95, and 99 weight percent.

As shown in the FIG. 1, the aqueous feedstock solution 1 is added to the reboiler 15 at a rate that provides sufficient residence time in the reaction zone 20 for complete or nearly complete conversion of sugars to furfural. The required residence time is a function of temperature and sugar concentration and is readily determined by one of skill in the art. In an embodiment, the residence time in the reaction zone is between about 1 s-500 min or 1-250 min or 5-120 min. In some embodiments, the residence time in the reaction zone is between and optionally including any two of the following values: 1 second, 5 seconds, 10 seconds, 30 seconds, 1 minute, 5 minutes, 10 minutes, 15 minutes, 20 minutes, 25 minutes, 30 minutes, 40 minutes, 60 minutes, 70 minutes, 80 minutes, 90 minutes, minutes, 100 minutes, 110 minutes, 120 minutes, 130 minutes, 150 minutes, 180 minutes, 210 minutes, 240 minutes, 300 minutes, 360 minutes, 420 minutes, 480 minutes, and 500 minutes. The feedstock solution 1 is added to the reaction zone 20 and the reaction mixture 22 formed by the aqueous acid solution 2 and the aqueous feedstock solution 1 is converted to a mixture 5 of furfural 8 and water 7 which is then partially vaporized and refluxes as part of the distillation column 10.

The temperature of the feedstock solution 1 in the reaction zone 20 is in the range of 90-250° C. or 120-220° C. or 150-200° C. In some embodiments, the temperature of the reaction mixture is between and optionally including any two of the following values: 90, 100, 110, 120, 130, 140, 150, 160, 170, 180, 190, 200, 210, 220, 230, 240, and 250° C.

The reaction is carried at a pressure between about atmospheric pressure and about 3.87 MPa, 0.5-3.4 MPa or 0.5-2.0 MPa.

The process further comprises removing the mixture 5 of water 7 and furfural 8 from the top of the reactive distillation column 10 and collecting reaction byproducts including water, unreacted sugars and nonvolatile byproducts 4 from the bottom of the reboiler 15, as shown in FIG. 1.

As the reaction proceeds, a mixture 5 of vapors of furfural 8 and water 7 are removed from the reaction mixture 22 via reflux through a multistage distillation column 10, condensed, and collected as a solution of furfural and water, as shown in FIG. 1. The use of staging in the distillation process allows more efficient stripping of furfural away from the acid catalyst solution. This increases furfural yield by driving the reaction toward completion and by minimizing formation of byproducts.

In addition the reactor configuration can also be equipped with a liquid purge capability to remove a portion (e.g., 20%) of the product flow exiting the reactor as liquid purge, preventing the buildup of high boilers (i.e. humins) in the reactor during operation.

The sugar in the feedstock undergoes chemical transformation to furfural, which, along with water (from the aqueous feedstock and water produced by the reaction), is then drawn at the top of the distillation column. This minimizes the residence time of furfural in the acidic environment and thereby minimizes its degradation. The furfural is separated from the water and purified by any convenient methods known in the art, and the product furfural is removed. The water is either recycled to the source of the feedstock sugar solution or is released from the process.

Reaction by-products such as water, unreacted sugars, and non-volatile byproducts such as humins are collected in the reboiler 15. The nonvolatile byproducts 4 are removed from the reboiler 15 (e.g., by filtration). The solution 6 of water and unreacted sugars can be disposed of, or at least a portion 8 can be fed as a stream 1 to be used as feedstock solution; if the solution is too dilute, it is concentrated by evaporation before being used as feedstock solution 1.

In one embodiment, with reference to FIG. 1, the reboiler 15 of the multistage column 10 is the reaction zone 20 and contains aqueous acid catalyst solution 2. A stream of aqueous sugar feedstock solution 1 is fed into the reboiler 15. A mixture 5 of furfural 8 and water 7 (as steam) are drawn off at the top of the column 10. Water, unreacted sugars, and nonvolatile byproducts (e.g., humins and other higher boiling byproducts) are collected in the reboiler 15. The nonvolatile materials 5 are removed from the reboiler 15. The remaining solution 6 in the reboiler is concentrated by evaporation, with evaporated water vapor 7 removed for disposal or reuse. The concentrated stream 8 is then fed back as feedstock solution 1.

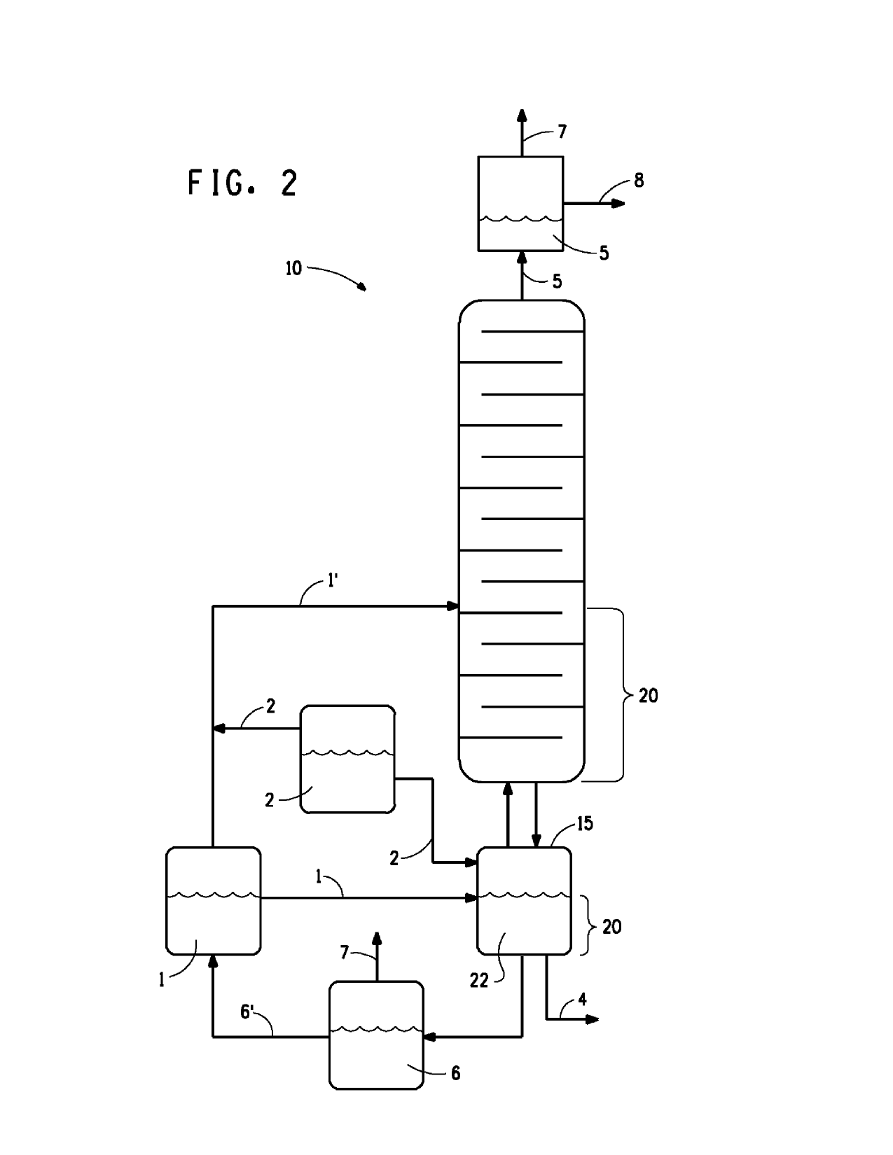

In an embodiment, the process further comprises adding the aqueous acid solution 2 to the aqueous feedstock solution 1 to form a premixed feedstock solution 1′ as shown in the FIG. 2, and feeding the premixed feedstock solution 1′ to the reboiler 15 through the reactive distillation column 10, thereby forming another reaction zone 20 in the reactive distillation column 10.

The process also comprises separating furfural 8 from the removed mixture 5 of water 7 and furfural 8, as shown in FIG. 1 and FIG. 2.

The process described above produces furfural from aqueous solutions of C₅ and/or C₆ sugars at both high yield and high conversion, without production of insoluble char in the reaction vessel. In an embodiment, the furfural yield is in the range of 40-95% or 60-95% or 65-85. In another embodiment, the furfural selectivity from sugar (C₅ sugar, C₆ sugar, or a mixture thereof) is in the range of 40-95% or 60-95% or 65-85%. In another embodiment, the conversion of sugar to furfural is in the range of 10-100% or 25-100% or 50-100%. Degradation of furfural is minimized by its low residence time in contact with the acid catalyst. Higher catalyst lifetime can be achieved because the catalyst is continually washed with the refluxing aqueous solution and not sitting in high-boiling byproducts like humins, which are known to be deleterious to catalyst lifetime.

In one embodiment, a process is provided comprising the steps of:

(a) providing a reactor comprising a multistage reactive distillation column comprising an upper, rectifying section; a lower, stripping section; and a reboiler, wherein the reboiler is a reaction zone containing an aqueous acid solution;

(b) continuously feeding an aqueous feedstock sugar solution comprising C₅ sugar, C₆ sugar or a mixture thereof to the reboiler, or to the column at any point, into contact with the aqueous acid solution, thereby forming a reaction mixture, wherein
(i) the temperature of the reaction mixture is between about 90° C. and about 250° C.
(ii) the reaction mixture is held at a pressure between about atmospheric pressure and about 3.87×10⁶ Pa, and
(iii) the feedstock sugar solution and aqueous acid catalyst solution are in contact for a time sufficient to produce water and furfural;

(c) drawing off a mixture of furfural and water at the top of the column;

(d) collecting water, unreacted sugars, and nonvolatile byproducts in the reboiler;

(e) removing nonvolatile byproducts from the reboiler; and

(f) removing the water and unreacted sugars from the reboiler for further use or disposal.

As used herein, where the indefinite article “a” or “an” is used with respect to a statement or description of the presence of a step in a process of this invention, it is to be understood, unless the statement or description explicitly provides to the contrary, that the use of such indefinite article does not limit the presence of the step in the process to one in number.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “invention” or “present invention is a non-limiting term and is not intended to refer to any single variation of the disclosed invention but encompasses all possible variations described in the specification and recited in the claims.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. The term “about” may mean within 10% of the reported numerical value, preferably within 5% of the reported numerical value.

### EXAMPLES

The methods described herein are illustrated in the following examples. From the above discussion and these examples, one skilled in the art can ascertain the essential characteristics of this invention, and without departing from the spirit and scope thereof, can make various changes and modifications of the invention to adapt it to various uses and conditions.

### Abbreviations

The meaning of abbreviations is as follows: “cm” means centimeter(s), “CSTR” means continuous stirred-tank reactor, “FF” means furfural, “g” means gram(s), “HPLC” means high pressure liquid chromatography, “min” means minute(s), “mL” means milliliter(s), “mm” means millimeter(s), “MPa” means megapascal(s), “psi” means pound(s) per square inch, “rpm” means revolutions per minute, “wt %” means weight percent(age), “μL” means microliter(s), and “μm” means micrometer(s).

### Materials

Xylose was obtained from Sigma-Aldrich Corporation (St. Louis, Mo.).

Deionized water was used unless otherwise indicated.

### Analytical Method

Furfural and sugar analysis was done by HPLC. Samples were collected and neutralized with calcium carbonate and then filtered through a 0.2 μm filter before it was analyzed by high pressure liquid chromatography (HPLC). The HPLC instrument employed was a HP 1100 Series equipped with Agilent 1200 Series refractive index (RI) detector and an auto injector (Santa Clara, Calif.). The analytical method was adapted from an NREL procedure (NREL/TP-510-42623). Separation and quantitation of monomeric sugars (glucose, xylose, and arabinose), and furfural (FF) was performed by injecting the sample (20 μL) on to a Bio-Rad HPX-87P (Bio-Rad, Hercules, Calif.) column maintained at 80° C. Water was used as the eluant, with a flowrate of 0.6 mL/min. The reaction products in the eluant were identified with the RI detector operating at 55° C.

### Example 1

### Conversion Via Aqueous Reactive Distillation of Sugar Solution to Furfural with Soluble Acid Catalyst

A 1000 mL zirconium 702 autoclave (Parr Corporation) was used as a Continuous Stirred-Tank Reactor (CSTR). The reactor was then connected to a continuous flow system capable of precisely controlled liquid feed. The reactor was heated by an electric heater which was temperature controlled to deliver excess heat to permit boilup of the solution in the reactor (internal and heater temperatures are read by 1/16″ (1.59 mm) stainless steel thermocouples). The vapor flow exiting the head of the reactor was then pressure controlled by a Grove Mighty-Mite backpressure regulator capable of more than a gage pressure of 500 psi (3.45 MPa) at the chosen vapor flows. The product from the regulator was then collected in sample vials for analysis by HPLC. The feed was 10 wt % xylose in water, fed to the liquid space of the autoclave by a Lewa Ecodos metering pump. The reactor heater was controlled in manual mode at 80% output (approximately 800 W heat, as heater is rated for 1000 W at full power) and the pressure was controlled at a gage pressure of 207 psi (1.43 MPa) by the backpressure regulator. The reactor was loaded with 560 grams of water and 0.56 grams of concentrated sulfuric acid (for about 0.1 wt % sulfuric acid) on startup. The reactor mixing was controlled at 500 rpm by an electric motor. Upon reaching temperature and pressure, the internal temperature stabilized at about 200° C., the distillate rate was measured and the feed rate adjusted to permit level control of the reactor by mass balance. At steady state conditions, about 2 hours after reaching the desired temperature and pressure, approximately 9.5 grams per minute of condensate was being removed from the vessel while about the same amount was fed continuously via the sugar feed pump. The condensate was sampled regularly through a water cooled condenser and analyzed for furfural composition by HPLC. The reaction was shut down after 5 hours at steady temperature and pressure, all feed and product flows stopped, and the heater shut off. The liquid in the reactor was found to contain significant solids byproducts, most likely humins produced by the reaction of furfural and xylose in acidic conditions. The yield to furfural was calculated by assuming a steady state in the reactor and evaluating the production of furfural as compared to the xylose feed. For example, with 10 wt % xylose feed, and no liquid purge from the reactor, a 100% yield to furfural would result in a furfural composition in the condensate of 10.0×96.08/150.1 (the ratio of the molecular weight of furfural to xylose) or 6.4 wt %. The results in table 1 below show the resulting furfural concentrations and calculated yield determined from the sampling of the experiment described above. The final column shows the measured composition of the reactor contents and their impact on process yield. Ideally the reactor in an experiment like this would be continuously purged to prevent the buildup of byproducts in the vessel. In addition, the feed of dilute sulfuric acid is desirable to make up for acid loss from purging and from acid loss reactions.

Table 1 below shows the result of a continuous run as described above. As used herein, the phrase “time on stream” means time since the start of feed to the reactor. The composition of furfural in the condensate appears to rapidly stabilize at about 4.2 wt % in slightly more than two hours of operation. This represents about a 64.1% yield to furfural from xylose. The final sample dropped somewhat to 3.9 wt % furfural or a yield of 60.4%. The final reactor composition was 2.92 wt % xylose.

TABLE 1
Furfural Production in Aqueous Reactive Distillation with Xylose Feed

| | Time on stream in minutes | | |
|---|---|---|---|
| | 76 | 142 | 191 |
| Xylose Feed, wt % | 9.77 | 9.77 | 9.77 |
| Average Condensate, wt % FF | 4.16 | 4.29 | 3.87 |
| Average Feed Rate, g/min | 9.96 | 9.92 | 9.80 |
| Xylose Feed, g/min | 0.97 | 0.97 | 0.96 |
| Furfural Production expected in | 0.62 | 0.62 | 0.61 |
| g/min @100% Yield | | | |
| Furfural Production, g/min | 0.399 | 0.406 | 0.370 |
| % FF Yield from Xylose | 64.1 | 65.4 | 60.4 |

### Example 2

### Conversion Via Aqueous Reactive Distillation of Sugar Solution to Furfural with Soluble Acid Catalyst, with Distillation Staging and Liquid Purge

The procedure used in Example 1 was followed in the same 1 Liter reactor equipped with a 1 inch (2.54 cm) packed column to allow staging of the distillation exiting the reactor for more efficient stripping of furfural from the reactor during reaction. In addition, the reactor was equipped with a liquid purge capability to remove approximately 20% of the product flow exiting the reactor as liquid purge preventing the buildup of high boilers (e.g., humins) in the reactor during operation. The feed mixture of xylose and water also contains makeup sulfuric acid (0.05 wt %) to balance the acid lost from the liquid purge. The reactor was run at the same conditions as in Example 1, 200 C internal temperature, gage pressure of 207 psi (1.43 MPa), 500 rpm stirring rate, with about 9.5 grams per minute feed rate of 10 wt % xylose and acid (0.05 wt %) and 7.2 grams per minute of distillate and 2.6 grams per minute of liquid purge from the reactor. Reflux in the 10 inches (25.4 cm) of 1 inch (2.54 cm) diameter structure packing above the column is accomplished via internal reflux caused by heat loss to the surroundings and is estimated at about 2 grams per minute. Purge was accomplished through manually dropping the level in the reactor via the rapid opening of a level control valve and tube every 15 minutes during the run. There was a slight loss of reactor temperature and pressure during this purge, but conditions were restored within a minute typically.

Table 2 shows the results of this continuous reactive distillation described above. Compared to example 1, the yield was lower owing to the loss of furfural and xylose in considerable quantities in the purge streams. If this material was completely recycled, the selectivity more closely represents what a true recycle steady state yield would be. The average selectivity to furfural from xylose is seen to be 59%, lower than the yield in the semi-batch operation in example 1. However, the buildup of humins in the reactor is considerably less thanks to the regular purging of the vessel which contained a slurry of humins and reactor liquid. The average concentration of the purge stream during the run was 2.4 wt % xylose and 1.0 wt % furfural.

TABLE 2
Continuous Furfural Production with Purge

| | Time in minutes since start of | | | |
|---|---|---|---|---|
| | xylose feed to reactor | | | |
| | 115 | 175 | 235 | 295 |
| Xylose Feed, wt % | 9.98% | 9.98% | 9.98% | 9.98% |
| Condensate rate, g/min | 7.65 | 7.25 | 6.94 | 7.13 |
| Average Condensate, wt % FF | 4.36 | 4.14 | 4.20 | 4.21 |
| Average Feed Rate, g/min | 9.57 | 9.56 | 9.44 | 9.50 |
| Xylose Feed, g/min | 0.96 | 0.95 | 0.94 | 0.95 |
| Furfural Production expected | 0.61 | 0.61 | 0.60 | 0.61 |
| g/min @100% yield | | | | |
| Furfural Production, including | 0.36 | 0.33 | 0.32 | 0.33 |
| purge, g/min | | | | |
| FF Yield from Xylose, Distillate | 54.5 | 49.1 | 48.3 | 49.5 |
| Only, % | | | | |
| FF Yield from Xylose, Distillate | 58.2 | 54.3 | 52.9 | 53.9 |
| and Purge % | | | | |
| Furfural Selectivity from Xylose, | 61.1 | 58.6 | 56.6 | 58.6 |
| Including Purge, % | | | | |
| Xylose Concentration Purge, | 1.90 | 2.23 | 2.53 | 2.88 |
| wt % | | | | |
| Furfural Concentration Purge, | 0.95 | 1.03 | 1.11 | 1.03 |
| wt % | | | | |
| Purge Flow Rate, g/min | 2.40 | 3.09 | 2.48 | 2.63 |
| Purge at % of Feed | 25.0 | 32.4 | 26.3 | 27.6 |

What is claimed is:

1. A process comprising:
(a) providing a reactor comprising a reactive distillation column disposed on top of a reboiler and an aqueous acid solution disposed in a reaction zone of the reboiler;
(b) bringing an aqueous feedstock solution into contact with the aqueous acid solution in the reaction zone for a residence time sufficient to produce a mixture of water and furfural, wherein the aqueous feedstock solution comprises C₅ sugar, C₆ sugar or a mixture thereof, and the reaction zone is at a temperature in the range of 90-250° C. and a pressure in the range of 0.1-3.87 M Pa;
(c) removing the mixture of water and furfural from the top of the reactive distillation column; and
(d) collecting water, unreacted sugars and nonvolatile byproducts from the reboiler.

2. The process according to claim 1 further comprises feeding the aqueous feedstock solution directly to the reboiler.

3. The process according to claim 1 further comprising:
e. adding the aqueous acid solution to the aqueous feedstock solution to form a premixed feedstock solution;
f. feeding the premixed feedstock solution to the reboiler through the reactive distillation column, thereby forming another reaction zone in the reactive distillation column.

4. The process according to claim 1 further comprising:
g. removing water and unreacted sugars from the water, unreacted sugars and nonvolatile byproducts of step (d); and
h. concentrating by evaporation at least a portion of the water and unreacted sugars and using it as aqueous feedstock solution in step (b).

5. The process according to claim 1 wherein the aqueous acid solution comprises a mineral acid, a heteropolyacid, an organic acid, or a combination thereof, present in a solvent at 0.01-10 weight %, based on total weight of the acid solution.

6. The process according to claim 2 wherein the acid catalyst is sulfuric acid.

7. The process according to claim 1 further comprising:
i. removing water and unreacted sugars from the water, unreacted sugars and nonvolatile byproducts of step (d); and
j. concentrating by evaporation at least a portion of the water and unreacted sugars and using it as aqueous feedstock solution in step (b).

8. The process according to claim 1 further comprising separating furfural from the removed mixture of water and furfural of step (c).

9. The process according to claim 1 wherein the combined concentration of C₅ sugar and/or C₆ sugar in the aqueous feedstock solution is in the range of 1-99 weight percent based on the total weight of the aqueous feedstock solution.

10. The process according to claim 9 wherein the combined concentration of C₅ sugar and/or C₆ sugar in the aqueous feedstock solution is in the range of 5-35 weight percent based on the total weight of the aqueous feedstock solution.

11. The process according to claim 1 wherein the aqueous feedstock solution comprises xylose, glucose, or a mixture thereof.

12. The process according to claim 1, further comprising removing a portion of the material in the reaction zone as liquid purge.

13. The process according to claim 1, wherein the reboiler is a continuous stirred-tank reactor.

14. A process comprising the steps of:
(a) providing a reactor comprising a multistage reactive distillation column comprising an upper, rectifying section; a lower, stripping section;
and a reboiler, wherein the reboiler is a reaction zone containing an aqueous acid solution;
(b) continuously feeding an aqueous feedstock sugar solution comprising C₅ sugar, C₆ sugar or a mixture thereof to the reboiler, or to the column at any point, into contact with the aqueous acid solution, thereby forming a reaction mixture, wherein
(i) the temperature of the reaction mixture is between 90° C. and 250° C.
(ii) the reaction mixture is held at a pressure between about atmospheric pressure and 400 psig, and
(iii) the feedstock sugar solution and aqueous acid catalyst solution are in contact for a time sufficient to produce water and furfural;
(c) drawing off a mixture of furfural and water at the top of the column;
(d) collecting water, unreacted sugars, and nonvolatile byproducts in the reboiler;
(e) removing nonvolatile byproducts from the reboiler; and
(f) removing the water and unreacted sugars from the reboiler for further use or disposal.
